SEQUENCE LISTING (6 sequences)

SEQ ID NO: 1 - DNA, 25 bp, Artificial Sequence
  Atrogin-1_F
cagtgatccattctgttcatccttg

SEQ ID NO: 2 - DNA, 25 bp, Artificial Sequence
  Atrogin-1_R
ttatttccagccaaatggagagaga

SEQ ID NO: 3 - DNA, 25 bp, Artificial Sequence
  MuRF-1_F
tctgcacttagaacacatagcagag

SEQ ID NO: 4 - DNA, 25 bp, Artificial Sequence
  MuRF-1_R
tctccttcttcattggtgttcttct

SEQ ID NO: 5 - DNA, 20 bp, Artificial Sequence
  b-actin_F
cagctcagtaacagtccgcc

SEQ ID NO: 6 - DNA, 20 bp, Artificial Sequence
  b-actin_R
tcactattggcaacgagcgg

(19) United States
(12) Patent Application Publication
(10) Pub. No.: US 2019/0183811 A1
(43) Pub. Date: Jun. 20, 2019

(54) COMPOSITION CONTAINING SQUALENE FOR IMPROVING MUSCLE FUNCTION AND PREVENTING MUSCLE DAMAGE

(71) Applicant: AAT COSTECH CO., LTD., Seoul (KR)

(72) Inventors: Jae Kwan HWANG, Seoul (KR); Se In LEE, Seoul (KR); Mi Bo KIM, Seoul (KR); Chang Hee KIM, Seoul (KR)

(73) Assignee: AAT COSTECH CO., LTD., Seoul (KR)

(21) Appl. No.: 16/322,825

(22) PCT Filed: Aug. 3, 2017
(86) PCT No.: PCT/KR2017/008397

§ 371 Date: Feb. 1, 2019

(30) Foreign Application Priority Data
Aug. 3, 2016 (KR) 10-2016-0098921
Aug. 3, 2017 (KR) 10-2017-0098409

(51) Int. Cl. A61K 31/01; A61P 21/00; A61K 9/16; A61K 9/20; A61K 9/48; A61K 9/00; A23L 33/10

(52) U.S. Cl. CPC A61K 31/01; A61P 21/00; A61K 9/1623; A61K 9/1611; A61K 9/2054; A23V 2002/00; A61K 9/2013; A61K 9/4866; A61K 9/0019; A23L 33/10; A61K 9/2018

(57) ABSTRACT

The present invention relates to a composition for preventing, treating, or ameliorating muscle disease or regenerating damaged muscles, comprising squalene. More specifically, the squalene of the present invention increases expression of proteins which are associated with muscle protein synthesis and muscle mass increase in muscle cells, inhibits, at an mRNA level, expression of enzymes involved in muscle protein degradation, and has an effect of rapidly restoring damaged muscles. In addition, the present invention relating to a natural product can be safely used without side effects, and thus can be used as medicines, foods, or cosmetics.

### TECHNICAL FIELD

The present application claims priority to Korean Patent Application No. 10-2016-0098921 filed on Aug. 3, 2016, and Korean Patent Application No. 10-2017-0098409 filed on Aug. 3, 2017, the contents of which are incorporated herein by reference in their entirety.

The present invention relates to a composition for preventing, treating, or ameliorating muscle disease and muscle injury, comprising squalene. More specifically, the present invention relates to a pharmaceutical composition for preventing or treating muscle disease and muscle damage, comprising squalene, a food composition for improving muscular function, preventing muscle damage, ameliorating muscle damage, or regenerating muscles, comprising squalene, and a cosmetic composition for improving muscular function or regenerating muscles, comprising squalene.

### BACKGROUND ART

Muscles are tissue formed by development of mesodermal stem cells and are composed of myofiber bundles caused by fusion of myoblasts. Muscles occupy 40% to 50% of a body weight, and support and protect bones and internal organs while, at the same time, allowing tissue other than muscles to have mobility as in heartbeat (Journal of Nutritional Science and Vitaminology, 61: 188-194, 2015). In addition to protecting organs, muscles greatly affect not only physical activity, including exercise, but also nutrient metabolism. Thus, muscles are also closely related to development of metabolic diseases such as type 2 diabetes, obesity, and cardiovascular disease.

Muscle atrophy is caused by a gradual decrease in muscle mass and refers to muscle weakness and degeneration (Cell, 119 (7): 907-910, 2004). Atrophy is promoted by inactivity, oxidative stress, or chronic inflammation, and weakens muscular function and motor ability (Clinical Nutrition, 26 (5): 524-534, 2007). The most important factor that determines muscular function is muscle mass, which is maintained by balance of protein synthesis and degradation. Muscular atrophy develops in a case where protein degradation occurs more than protein synthesis (The International Journal of Biochemistry and Cell Biology, 37 (10): 1985-1996, 2005).

Muscle size is regulated by intracellular signaling pathways that induce anabolism or catabolism which occurs within muscles. Muscle protein synthesis is increased in a case where signaling reactions that induce muscle protein synthesis occur more than those that induce muscle protein degradation. Such increased muscle protein synthesis is presented by increased muscle size (hypertrophy) and increased number of myofibers (hyperplasia) due to increase in muscle protein (The Korea Journal of Sports Science, 20 (3): 1551-1561, 2011).

Factors involved in muscle protein synthesis phosphorylate downstream proteins starting from stimulation of the phosphatidylinositol-3 kinase (PI3K)/Akt pathway in muscle cells, and thus induce protein synthesis. Activity of the mammalian target of rapamycin (mTOR) caused by PI3K/Akt signaling is recognized as a central growth signaling factor that integrates various growth signals in cells. mTOR activates 4E-binding protein (4EBP1) and phosphorylated 70-kDa ribosomal S6 kinase (p70S6K), which are two factors that initiate mRNA translation, and thus induces muscle protein synthesis, thereby contributing to increased muscle mass (The Korea Journal of Sports Science, 20 (3): 1551-1561, 2011; The International Journal of Biochemistry and Cell Biology, 43 (9): 1267-1276, 2011). Conversely, in a case where forhead box (FoxO), which is a transcription factor, migrates from cytoplasm into nucleus, FoxO increases expression of atrogin-1 and MuRF1 which are E3 ubiquitin ligase factors and involved in protein degradation (Disease Models and Mechanisms, 6: 25-39, 2013). Increased expression levels of atrogin-1 and MuRF1 promote protein degradation in muscles, which results in decreased muscle mass. Thus, promoted activity of mTOR and inhibited expression of atrogin-1 and MuRF1 increase an amount of muscle proteins and lead to increased muscle mass.

In a case where there is injury or damage in muscles, muscle satellite cells which are precursors to muscle cells play an important role in muscle regeneration. In a case where muscles are in a normal state, the muscle satellite cells located at the edge of myofibers remain quiescent. However, in a case where muscles are physically or chemically damaged from an outside, various transcription factors are secreted to regenerate the damaged muscles and a muscle regeneration step begins.

Transcription factors expressed due to muscle injury cause the satellite cells to undergo self-renewal and form a satellite cell pool to be used for muscle regeneration. Here, expression of pax7 is increased in order that the number of the satellite cells required for muscle regeneration is kept constant. In a case where the transcription factor pax7 with increased expression is methylated by carm1 protein, a pax7-carm1 complex is formed to promote expression of myf5. In a case where muscle satellite cells in a quiescent state are activated by increased expression level of the myf5 factor, the activated muscle satellite cells migrate to injured and damaged muscle sites so that myofiber bundles are formed and new muscles are produced (Stem Cells Translational. Medicine, 5: 282-290, 2016).

Squalene is an unsaturated hydrocarbon in which 30 carbon atoms and 50 hydrogen atoms are linked by 6 double bonds. Squalene is widely, although in a small amount, distributed in a human body, and animal and plant kingdoms, and, in particular, is abundant in deep-sea sharks. For physiochemical actions of squalene, it has been reported that squalene has activity of ameliorating hypertriglyceridemia through a lipid metabolic process (European Journal of Lipid Science and Technology, 118: 1-7, 2016), antioxidative and antitumor activity (The Lancet Oncology, 1: 107-112, 2000), activity against breast cancer (Food and Chemical Toxicology, 48: 1092-1100, 2010), atherosclerosis, hyperlipidemia, and liver steatosis (Biotechnology Letters, 38: 1065-1071, 2016), and the like. In addition, squalene is used as a lubricant for cosmetics and computer disks. However, nothing is known about prevention and treatment of muscle disease by squalene, or improvement of muscular function by squalene.

Accordingly, the present inventors have searched for a natural substance that can be safely applied while having superior activity of regulating muscular function. As a result, the present inventors have identified that squalene can increase expression of proteins which are associated with muscle protein synthesis and muscle mass increase in muscle cells, inhibit, at an mRNA level, expression of enzymes involved in muscle protein degradation, and rapidly restore damaged muscles, so that the squalene of the present invention can be used as an active ingredient of a composition for preventing, treating, or ameliorating muscle disease and muscle damage, and therefore have completed the present invention.

### Technical Problem

Accordingly, the present inventors have searched for a natural substance that can be safely applied while having superior activity of regulating muscular function. As a result, the present inventors have identified that squalene can increase muscle mass and improve muscular function, and can exhibit an effect of ameliorating and preventing muscle damage, and therefore have completed the present invention.

Therefore, an object of the present invention is to provide a pharmaceutical composition for preventing or treating muscle disease or muscle damage.

Another object of the present invention is to provide a food composition for improving muscular function, preventing muscle damage, ameliorating muscle damage, or regenerating muscles.

Still another object of the present invention is to provide a cosmetic composition for improving muscular function or regenerating muscles.

### Solution to Problem

In order to achieve the above objects, the present invention provides a pharmaceutical composition for preventing or treating muscle disease or muscle damage, comprising squalene as an active ingredient.

In addition, the present invention provides a food composition for improving muscular function, preventing muscle damage, ameliorating muscle damage, or regenerating muscles, comprising squalene as an active ingredient.

In addition, the present invention provides a cosmetic composition for improving muscular function or regenerating muscles, comprising squalene as an active ingredient.

### Advantageous Effects of Invention

Accordingly, the present invention provides a composition for preventing, treating, or ameliorating muscle disease and muscle damage, comprising squalene as an active ingredient.

The squalene of the present invention can increase expression of proteins which are associated with muscle protein synthesis and muscle mass increase in muscle cells, inhibit, at an mRNA level, expression of enzymes involved in muscle protein degradation, and rapidly restore damaged muscles. Thus, the squalene of the present invention increases muscle mass, so that muscular function can be improved and an effect of ameliorating and preventing muscle damage can be exhibited, which allows the squalene to be effectively used as an active ingredient of a composition for preventing, treating, or ameliorating muscle disease and muscle damage.

### BRIEF DESCRIPTION OF DRAWINGS

FIG. 1 illustrates a protein expression level of p-mTOR in C2C12 muscle cells following treatment with squalene.

FIG. 2 illustrates a protein expression level of p-p70S6K in C2C12 muscle cells following treatment with squalene.

FIG. 3 illustrates mRNA expression levels of atrogin-1 and MuRF1 in C2C12 muscle cells following treatment with squalene.

FIG. 4 illustrates muscular strength of experimental animals following treatment with squalene.

FIG. 5 illustrates a weight increase in the right tibialis anterior muscle of experimental animals following treatment with squalene.

FIG. 6 illustrates a myofiber cross-sectional area in the right tibialis anterior muscle following treatment with squalene.

FIG. 7 illustrates a weight increase in the right gastrocnemius muscle of experimental animals following treatment with squalene.

### DETAILED DESCRIPTION OF INVENTION

Hereinafter, the present invention will be described in detail.

As described above, search for a substance that can be safely applied while having superior activity of regulating muscular function is continuously required. However, studies about effects of squalene on prevention, treatment, or amelioration of muscle disease and muscle damage have not yet been reported.

The squalene of the present invention can increase expression of proteins which are associated with muscle protein synthesis and muscle mass increase in muscle cells, inhibit, at an mRNA level, expression of enzymes involved in muscle protein degradation, and rapidly restore damaged muscles, and thus is effectively used as an active ingredient of a composition for preventing, treating, or ameliorating muscle disease and muscle damage.

Accordingly, the present invention provides a pharmaceutical composition for preventing or treating muscle disease or muscle damage, comprising, as an active ingredient, squalene represented by a structure the following [Formula 1]:

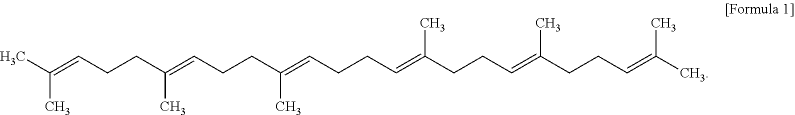 [SMILES: CC(C)=CCC/C(C)=C/CC/C(C)=C/CC/C=C(\C)CC/C=C(\C)CCC=C(C)C]

In addition, the present invention provides a food composition for improving muscular function, preventing muscle damage, ameliorating muscle damage, or regenerating muscles, comprising the squalene as an active ingredient.

In addition, the present invention provides a cosmetic composition for improving muscular function or regenerating muscles, comprising the squalene as an active ingredient.

The squalene can be represented by Chemical Abstracts Service Number (CAS No.) 111-02-04, and a structural name thereof is (6E,10E,14E,18E)-2,6,10,15,19,23-hexamethyltetracosa-2,6,10,14,18,22-hexaene. As the squalene of the present invention, any of those obtained by isolation from extracts, obtained by synthesis, or obtained from commercially available products may be used.

As used herein, the term “muscle disease” is preferably a disease reported in the art as muscle disease caused by decreased muscular function, muscle wasting, or muscle degeneration. Specifically, the muscle disease is more preferably, but not limited to, any one or more selected from the group consisting of sarcopenia, muscular atrophy, muscular dystrophy, muscle degeneration, and cachexia.

The muscle wasting or muscle degeneration occurs due to genetic factors, acquired factors, aging, or the like. The muscle wasting is characterized by gradual loss of muscle mass, and weakness and degeneration of muscles, in particular, skeletal muscles or voluntary muscles, and heart muscles.

In addition, as used herein, the term “muscle damage” refers to damage caused by physical or chemical destruction due to a wound, and is more preferably, but not limited to, any one or more selected from the group consisting of muscle strain, muscle rupture, muscle tearing, contusion, distortion, rotator cuff syndrome, and myositis.

The physical destruction occurs due to trauma, excessive temperature, myotoxin, ischemia, inflammation, exercise, or the like, and is characterized by damage to skeletal muscles or voluntary muscles, and heart muscles.

In addition, as used herein, the term “regenerating muscles” means rapidly restoring muscles in a case where the muscles are physically or chemically damaged, and refers collectively to a process and a period required until the damaged muscles can perform a normal function.

More specifically, the term “muscle” refers collectively to sinew, muscle, and tendon. The term “muscular function” or “muscle function” means an ability to exert force by contraction of muscles, and includes muscular strength which is an ability of muscles to exert maximum contraction force to overcome resistance; muscle endurance which is an ability of muscles indicating how long or how many times the muscles can repeat contraction and relaxation against a given weight; and explosive muscular strength which is an ability of muscles to exert strong force in a short period of time. These muscular functions are managed by the liver and are proportional to muscle mass.

The term “improving muscular function” means improving muscular function in a more positive direction. Specifically, the above-mentioned “improving muscular function” means that as squalene is administered, muscle proteins are synthesized to increase muscle mass, which can induce an effect of regenerating muscles and makes it possible to expect that muscle damage is prevented or ameliorated, so that an effect of improving muscular function can be exhibited.

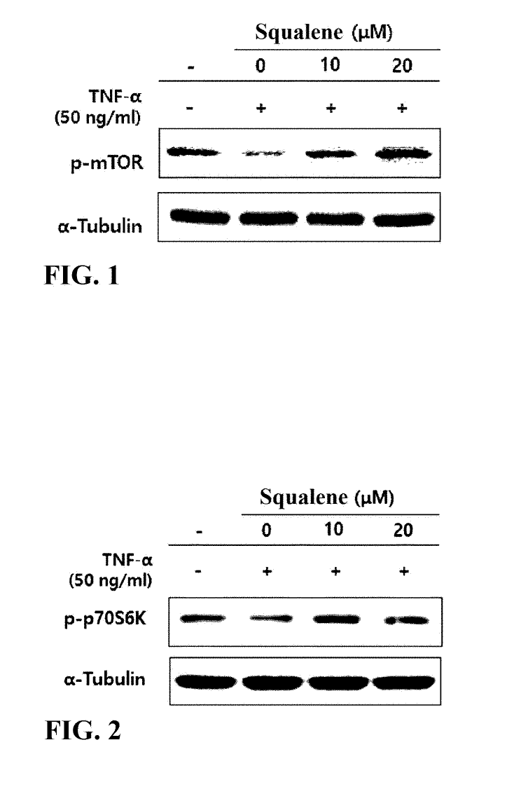

In a specific embodiment of the present invention, the present inventors identified that squalene increases expression of proteins, which are associated with muscle protein synthesis and muscle mass increase, in muscle cells (FIGS. 1 and 2).

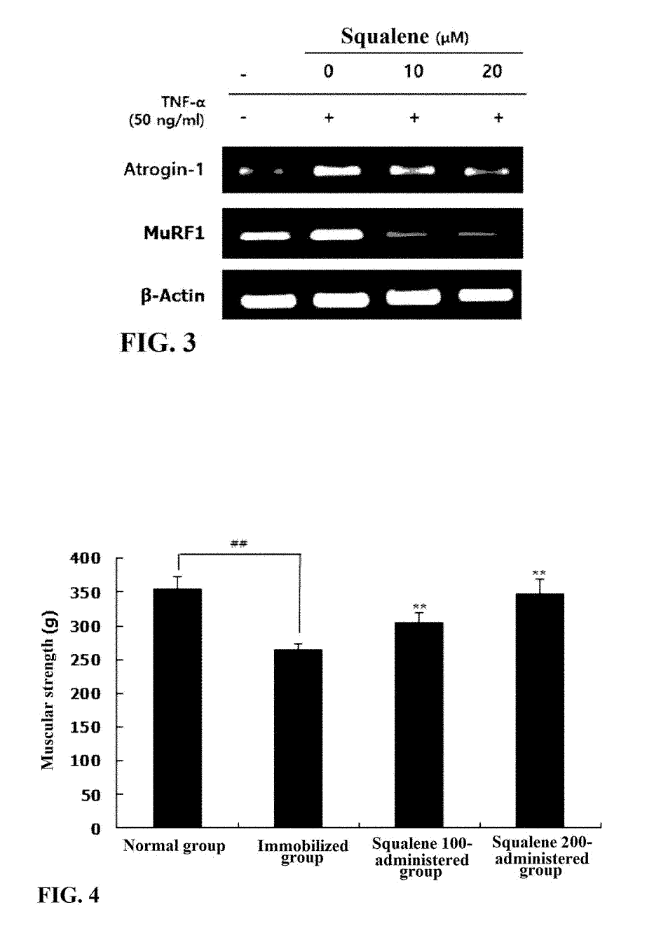

In another specific embodiment of the present invention, the present inventors identified that squalene decreases, at an mRNA level, expression of MuRF1 and atrogin-1, which are enzymes involved in muscle protein degradation, in muscle cells (FIG. 3).

In still another specific embodiment of the present invention, the present inventors identified that squalene increases decreased muscular strength, muscle weight, and myofiber cross-sectional area in immobilized experimental animals (FIGS. 4, 5, and 6).

In still yet another specific embodiment of the present invention, the present inventors identified that squalene rapidly restores damaged muscles in immobilized experimental animals (FIG. 7).

Therefore, the squalene of the present invention can increase expression of proteins which are associated with muscle protein synthesis and muscle mass increase in muscle cells, inhibit, at an mRNA level, expression of enzymes involved in muscle protein degradation, and rapidly restore damaged muscles, and thus can be used as an active ingredient of a pharmaceutical composition for preventing or treating muscle disease and muscle damage.

The composition for preventing or treating muscle disease and muscle damage of the present invention may contain squalene alone or in combination with at least one active ingredient which exhibits a similar function to squalene. In a case where an additional ingredient is contained, the composition of the present invention may exhibit a further enhanced muscular function-improving effect. When the above ingredient is additionally used, skin safety, easiness of formulation, and stability of active ingredients due to such combined use should be taken into consideration.

The pharmaceutical composition of the present invention may comprise a pharmaceutically acceptable salt of squalene. As used herein, the term “pharmaceutically acceptable” refers to being physiologically acceptable and typically not causing an allergic reaction or a similar reaction in a case of being administered to a human. The pharmaceutically acceptable salt is preferably an acid addition salt formed with a pharmaceutically acceptable free acid.

The pharmaceutically acceptable salt of squalene may be an acid addition salt formed with an organic acid or an inorganic acid. Examples of the organic acid include formic acid, acetic acid, propionic acid, lactic acid, butyric acid, isobutyric acid, trifluoroacetic acid, malic acid, maleic acid, malonic acid, fumaric acid, succinic acid, succinic acid monoamide, glutamic acid, tartaric acid, oxalic acid, citric acid, glycolic acid, glucuronic acid, ascorbic acid, benzoic acid, phthalic acid, salicylic acid, anthranilic acid, dichloroacetic acid, aminooxyacetic acid, benzene sulfonic acid, p-toluenesulfonic acid, and methanesulfonic acid. Examples of the inorganic acid include hydrochloric acid, bromic acid, sulfuric acid, phosphoric acid, nitric acid, carbonic acid, and boric acid. The acid addition salt may be preferably in the form of hydrochloride or acetate, and may be more preferably in the form of hydrochloride.

The above-mentioned acid addition salt is prepared using common methods for preparing a salt, such as a) performing direct mixing of squalene and an acid, b) dissolving the squalene and the acid in a solvent or a water-containing solvent, and then performing mixing, and c) placing squalene in an acid in a solvent or a hydrated solvent, and then performing mixing. In addition to the above, additional possible salt forms include GABA salts, gabapentin salts, pregabalin salts, nicotinates, adipates, hemimalonates, cysteine salts, acetylcysteine salts, methionine salts, arginine salts, lysine salts, ornithine salts, aspartates, and the like.

In addition, the pharmaceutical composition of the present invention for preventing or treating muscle disease and muscle damage may further comprise a pharmaceutically acceptable carrier.

As the pharmaceutically acceptable carrier, for example, a carrier for oral administration or a carrier for parenteral administration may be additionally included. The carrier for oral administration may include lactose, starch, cellulose derivatives, magnesium stearate, stearic acid, and the like. In addition, the carrier for parenteral administration may additionally include water, suitable oil, saline, aqueous glucose, glycol, and the like. In addition, a stabilizer or a preservative may be additionally contained. Suitable stabilizers include antioxidants such as sodium hydrogen sulfite, sodium sulfite, and ascorbic acid. Suitable preservatives include benzalkonium chloride, methyl- or propyl-paraben, and chlorobutanol. For the other pharmaceutically acceptable carriers, reference can be made to those described in the following literature (Remington's Pharmaceutical Sciences, 19th ed., Mack Publishing Company, Easton, Pa., 1995).

The pharmaceutical composition of the present invention may be administered in any way to a mammal including a human. For example, the pharmaceutical composition may be administered orally or parenterally. Parenteral administration methods may include, but are not limited to, intravenous administration, intramuscular administration, intraarterial administration, intramedullary administration, intradural administration, intracardiac administration, transdermal administration, subcutaneous administration, intraperitoneal administration, intranasal administration, enteral administration, topical administration, sublingual administration, and rectal administration.

The pharmaceutical composition of the present invention may be formulated into a preparation for oral administration or parenteral administration depending on the route of administration as described above. In a case of being formulated into the preparation, preparation can be made using one or more of buffers (for example, saline or PBS), antioxidants, bacteriostatic agents, chelating agents (for example, EDTA or glutathione), fillers, extenders, binders, adjuvants (for example, aluminum hydroxide), suspending agents, thickeners, wetting agents, disintegrants or surfactants, and diluents or excipients.

Solid preparations for oral administration include tablets, pills, powders, granules, liquids, gels, syrups, slurries, suspensions, capsules, and the like. Such solid preparations may be prepared by mixing the pharmaceutical composition of the present invention with at least one excipient such as starch (including corn starch, wheat starch, rice starch, potato starch, and the like), calcium carbonate, sucrose, lactose, dextrose, sorbitol, mannitol, xylitol, erythritol, maltitol, cellulose, methyl cellulose, sodium carboxymethyl cellulose, hydroxypropyl methylcellulose, and gelatin. For example, tablets or sugarcoated tablets may be obtained by blending an active ingredient with a solid excipient, grinding the blend, adding a suitable adjuvant thereto, and then processing the resultant into a granule mixture.

In addition to simple excipients, lubricants such as magnesium stearate and talc are also used. Liquid preparations for oral use include suspensions, solutions, emulsions, syrups, and the like. In addition to water or liquid paraffin, which is a commonly used simple diluent, the liquid preparations may contain various excipients such as a wetting agent, a sweetener, a fragrance, and a preservative.

In addition, in some cases, crosslinked polyvinylpyrrolidone, agar, alginic acid, sodium alginate, or the like may be added as a disintegrant. An anticoagulant, a lubricant, a wetting agent, a flavoring agent, an emulsifying agent, an antiseptic agent, or the like may be further contained.

In a case of being administered parenterally, the pharmaceutical composition of the present invention may be formulated, along with a suitable parenteral carrier, in the form of an injection, a transdermal preparation, and a nasal inhaler according to a method known in the art. The injection must be sterilized and protected against contamination of microorganisms such as bacteria and fungi. In a case of the injection, examples of suitable carriers may include, but not limited to, solvents or dispersion media which contains water, ethanol, polyol (for example, glycerol, propylene glycol, and liquid polyethylene glycol), mixtures thereof, and/or vegetable oil. More preferably, as the suitable carriers, an isotonic solution such as Hank's solution, Ringer's solution, triethanolamine-containing phosphate buffered saline (PBS) or sterilized water for injection, 10% ethanol, 40% propylene glycol, and 5% dextrose, or the like may be used. In order to protect the injection against contamination of microorganisms, various antibacterial agents and antifungal agents such as paraben, chlorobutanol, phenol, sorbic acid, and thimerosal may be further contained. In addition, in most cases, the injection may further contain an isotonic agent such as sugar or sodium chloride.

The preparation for transdermal administration may take forms such as an ointment, a cream, a lotion, a gel, a liquid for external use, a paste, a liniment, and an aerosol. In this case, “transdermal administration” means administering a pharmaceutical composition topically to skin so that an effective amount of an active ingredient contained in the pharmaceutical composition is delivered into the skin.

In a case of a preparation for inhaler administration, a compound to be used according to the present invention may be conveniently delivered in the form of an aerosol spray from a pressurized pack or a nebulizer, using a suitable propellant such as dichlorofluoromethane, trichlorofluoromethane, dichlorotetrafluoroethane, carbon dioxide, or another suitable gas. In a case of a pressurized aerosol, a unit dosage may be determined by providing a valve that delivers a metered amount. For example, gelatin capsules and cartridges for use in an inhaler or insufflator may be formulated to contain a powder mixture of a compound and a suitable powder base such as lactose and starch. Preparations for parenteral administration are described in Remington's Pharmaceutical Science, 15th Edition, 1975, Mack Publishing Company, Easton, Pa. 18042, Chapter 87: Blaug, Seymour, which is a prescription manual commonly known in all pharmaceutical chemistries.

The pharmaceutical composition of the present invention for preventing or treating muscle disease and muscle damage can provide desired effects of preventing or treating the muscle disease and muscle damage in a case where squalene is contained in an effective amount. As used herein, the term “effective amount” refers to an amount that results in a higher response than a negative control, and preferably refers to an amount sufficient to improve muscular function. In the pharmaceutical composition of the present invention, squalene may be contained in an amount of 0.01% to 99.99%, and the remainder is occupied by a pharmaceutically acceptable carrier. An effective amount of squalene contained in the pharmaceutical composition of the present invention may vary depending on a product form of the composition, and the like.

A total effective amount of the pharmaceutical composition of the present invention may be administered to a patient as a single dose, or as multiple doses by a fractionated treatment protocol intended for a long-term administration. It is important to administer an amount such that a maximum effect can be obtained with a minimum amount without side effects by taking all of the above-described factors into consideration, and such an amount can be readily determined by those skilled in the art.

A content of an active ingredient in the pharmaceutical composition of the present invention may vary depending on severity of a disease. The pharmaceutical composition may be administered as a single dose or in divided doses such that the squalene is administered in an amount of preferably 0.01 to 50 mg and more preferably 0.1 to 30 mg per kg body weight a day in a case of parenteral administration, and such that the squalene is administered in an amount of preferably 0.01 to 100 mg and more preferably 0.01 to 10 mg per kg body weight a day in a case of oral administration. However, for a dosage of the squalene, an effective dose for a patient is determined in consideration of not only route of administration for the pharmaceutical composition and frequency of treatment but also various factors such as the patient's age, body weight, health condition, and sex, severity of a disease, a diet, and an excretion rate. Thus, in view of this, a person of ordinary skill in the art would be able to determine a suitable effective dose for the squalene depending on particular uses for prevention and treatment of muscle disease. For the pharmaceutical composition according to the present invention, there is no particular limitation on formulation, route of administration, and administration method as long as an effect of the present invention is exhibited.

The pharmaceutical composition of the present invention for preventing or treating muscle disease and muscle damage may be used either alone or in combination with methods which use surgery, radiation therapy, hormonal therapy, chemotherapy, or biological response modifiers.

The pharmaceutical composition of the present invention for preventing or treating muscle disease and muscle damage may also be provided as a preparation for external use, comprising squalene as an active ingredient.

In a case where the pharmaceutical composition of the present invention for preventing or treating muscle disease and muscle damage is used as an external preparation for skin, the pharmaceutical composition may further contain adjuvants commonly used in the field of dermatology such as any other ingredients commonly used for the external preparation for skin including a fatty substance, an organic solvent, a solubilizing agent, a concentrating agent and a gelling agent, a softening agent, an antioxidant, a suspending agent, a stabilizing agent, a foaming agent, a fragrance, a surfactant, water, an ionic emulsifying agent, a nonionic emulsifying agent, a filling agent, a metal ion blocking agent, a chelating agent, a preservative, a vitamin, a blocking agent, a wetting agent, essential oil, a dye, a pigment, a hydrophilic activator, a lipophilic activator, a lipid vesicle, and the like. In addition, the above ingredients may be introduced in an amount commonly used in the field of dermatology.

In a case where the pharmaceutical composition of the present invention for preventing or treating muscle disease and muscle damage is provided as an external preparation for skin, the pharmaceutical composition may be, but not limited to, a preparation such as an ointment, a patch, a gel, a cream, and a spray.

In addition, the present invention can be used as an active ingredient of a food composition for preventing or ameliorating muscle disease or muscle damage, comprising squalene as an active ingredient.

The food composition of the present invention includes all forms such as functional foods, nutritional supplements, health foods, food additives, and animal foods, and is intended for feeding animals including a human and a domesticated animal. Food compositions of such types can be prepared in various forms according to conventional methods known in the art.

The food composition according to the present invention can be prepared in various forms according to conventional methods known in the art. General foods may be prepared by adding the squalene of the present invention to beverages (including alcoholic beverages), fruits and foods processed therefrom (for example, canned fruit, bottled fruit, jam, and marmalade), fishes, meats and foods processed therefrom (for example, ham, sausage, and corn beef), bread and noodles (for example, udon, buckwheat noodles, ramen, spaghetti, and macaroni), juices, various drinks, cookies, taffies, dairy products (for example, butter and cheese), edible vegetable oil and fat, margarine, vegetable proteins, retort foods, frozen foods, various seasonings (for example, soybean paste, soy sauce, and sauces), or the like, but preparation methods are not limited thereto. In addition, the nutritional supplement may be prepared by adding the squalene of the present invention to capsules, tablets, pills, or the like, but preparation methods are not limited thereto. In addition, the squalene of the present invention can be liquefied, granulated, encapsulated, or powdered, and ingested, by preparing the squalene of the present invention itself in the form of tea, a juice, or a drink so as to be drinkable (health beverage). In addition, in order to use the squalene of the present invention in the form of a food additive, the squalene can be prepared in the form of a powder or a concentrate and used. In addition, the squalene of the present invention may be mixed together with an active ingredient known to be effective for prevention or amelioration of muscle disease and muscle damage so as to be prepared in the form of a composition.

In a case where the squalene of the present invention is used for a health beverage, such a health beverage composition may contain, as additional ingredients, various flavoring agents, natural carbohydrates, or the like as in ordinary beverages. The above-mentioned natural carbohydrate may be a monosaccharide such as glucose and fructose; a disaccharide such as maltose and sucrose; a polysaccharide such as dextrin and cyclodextrin; or sugar alcohol such as xylitol, sorbitol, and erythritol. As the sweetening agent, a natural sweetening agent such as thaumatin and a stevia extract; a synthetic sweetening agent such as saccharin and aspartame, or the like may be used. A proportion of the natural carbohydrate is generally about 0.01 to 0.04 g and preferably about 0.02 to 0.03 g, per 100 mL of the composition of the present invention.

In addition, the squalene of the present invention may be contained as an active ingredient of a food composition for preventing or ameliorating muscle disease and muscle damage. An amount of the squalene is an amount effective for achieving action of preventing muscle disease and improving muscular function. The amount is not particularly limited and is preferably 0.01% to 100% by weight with respect to a total weight of the entire composition. The food composition of the present invention may be prepared by mixing squalene together with other active ingredients known to be effective for preventing or ameliorating muscle disease and muscle damage.

In addition to the above, the health food of the present invention may further contain various nutrients, vitamins, electrolytes, flavoring agents, colorants, pectic acid, salts of pectic acid, alginic acid, salts of alginic acid, organic acids, protective colloids, thickeners, pH adjusting agents, stabilizers, preservatives, glycerin, alcohol, carbonating agents, or the like. In addition, the health food of the present invention may further contain fruit flesh for preparing a natural fruit juice, a fruit juice beverage, or a vegetable beverage. These ingredients may be used independently or in admixture. A proportion of such additives is not critical, and is generally selected in a range of 0.01 to 0.1 parts by weight per 100 parts by weight of the composition of the present invention.

In addition, the present invention provides a cosmetic composition for improving muscular function or promoting muscle regeneration, comprising squalene as an active ingredient.

The cosmetic composition is not particularly limited, and may be used for external use on skin or may be ingested orally.

The cosmetic composition of the present invention comprises squalene as an active ingredient, and may be prepared, together with dermatologically acceptable excipients, in the form of basic cosmetic compositions (face cleansing agents such as lotion, cream, essence, cleansing foam, and cleansing water, pack, and body oil), color cosmetic compositions (foundation, lipstick, mascara, and makeup base), hair product compositions (shampoo, rinse, hair conditioner, and hair gel), soaps, and the like.

Examples of such excipients may include, but are not limited to, skin emollients, skin penetration enhancers, colorants, fragrances, emulsifiers, concentrating agents, and solvents. In addition, flavoring agents, pigments, bactericides, antioxidants, preservatives, moisturizers, and the like may be further contained. For the purpose of improving physical properties, thickeners, inorganic salts, synthetic polymeric substances, and the like may be further contained. For example, in a case where a face cleansing agent and a soap are prepared with the cosmetic composition of the present invention, preparation can be easily made by adding the squalene to common bases for the face cleansing agent and the soap. In a case of preparing the cream, preparation may be made by adding the squalene or a salt thereof to a typical oil-in-water (O/W) cream base. To this may be further added a flavoring agent, a chelating agent, a pigment, an antioxidant, a preservative, and the like as well as a synthetic or natural material, such as a protein, a mineral, and a vitamin, which is intended to improve physical properties.

A content of squalene contained in the cosmetic composition of the present invention is, but not limited to, preferably 0.001% to 10% by weight, and more preferably 0.01% to 5% by weight, with respect to a total weight of the entire composition. In a case where the content is less than 0.001% by weight, a desired anti-aging or wrinkle-improving effect cannot be expected. In a case where the content is more than 10% by weight, it may be difficult to maintain safety or to formulate preparations.

Hereinafter, the present invention will be described in more detail with reference to examples and preparation examples. It should be apparent to those skilled in the art that these examples and preparations are merely for illustrating the present invention and that the scope of the present invention is not construed as being limited by these examples and preparation examples.

### Example 1

Effect of Squalene which Increases Phosphorylation Level of mTOR Protein in Muscle Cells

In a case where mTOR protein is activated by phosphorylation, expression levels of major proteins in muscle cells which are expressed upon differentiation and are associated with muscle protein synthesis and muscle mass increase in a PI3K/Akt signaling pathway were checked. The obtained cells were lysed with an NP-40 buffer solution (ELPISBIOTECH. INC, Daejeon, Korea) containing a protease inhibitor cocktail (Sigma-Aldrich), and centrifuged at 13,000 rpm for 10 minutes to obtain a cell lysate which is a supernatant. A protein concentration in the supernatant was quantitated by Bradford. Then, a predetermined concentration of the proteins was heated for 5 minutes and separated by SDS-PAGE electrophoresis. The separated proteins were transferred to a nitrocellulose membrane. Then, a p-mTOR primary antibody (Cell Signaling Technology, Inc., Beverly, Mass., USA) was diluted with 2.5% bovine serum albumin (BSA; BioWORLD, Dublin, Ohio, USA) at a ratio of 1:1,000, and the resultant was allowed to react with the proteins, which had been transferred to the nitrocellulose membrane, at room temperature for 20 hours. After the reaction with the primary antibody, the nitrocellulose membrane was washed three times for 10 minutes using Tris-buffer Saline Tween 20 (TBST). After performing washing, an anti-rabbit secondary antibody (Bethyl Laboratories, Inc., Montgomery, TA, USA) to which horseradish peroxidase had been conjugated and which recognizes the primary antibody, was diluted with 2.5% BSA (BioWORLD) so as to reach 1:5,000, and the resultant was allowed to react with the nitrocellulose membrane at room temperature for 2 hours. The nitrocellulose membrane was washed three times for 10 minutes each using TBST. Protein bands detected through antibody binding were developed using the ECL Western Blot Detection Reagent (Amersham, Tokyo, Japan), and the developed protein bands were identified using a G:BOX EF imaging system (Syngene, Cambridge, UK).

As a result, as illustrated in FIG. 1, it was identified that an expression level of phosphorylated mTOR (p-mTOR) is increased in C2C12 muscle cells due to treatment with squalene. This means that squalene has a superior effect of increasing muscle production in muscle cells.

### Example 2

Effect of Squalene which Promotes mRNA Translation Activity in Muscle Cells

It had been identified that squalene exhibits an effect of increasing muscle production in muscle cells. Thus, in order to identify this fact in a more specific manner, a phosphorylation-induced activity level of p70S6K protein, which is known to be involved in an mRNA translation process in muscle cells, was checked, instead of an expression level of p-mTOR.

Specifically, C2C12 muscle cells were treated with squalene and cultured while inducing differentiation, by performing the same method as in the above <Example 1>. The resulting cells were obtained and subjected to western blotting. A p-p70S6K antibody (Santa Cruz Biotechnology, Inc., Santa Cruz, Calif., USA) was used as a primary antibody for western blotting.

As a result, as illustrated in FIG. 2, it was identified that an expression level of the p-p70S6K protein is increased in C2C12 muscle cells due to treatment with squalene. This means that squalene has a superior ability to promote the mRNA translation process for muscle production in muscle cells.

### Example 3

Effect of Squalene which Exhibits Inhibitory Activity on Muscle Protein Degradation in Muscle Cells

It had been identified that squalene exhibits an effect of increasing muscle production in muscle cells. Thus, in order to identify whether the produced muscle proteins can also be protected against degradation through degradation-inhibitory activity of squalene, mRNA transcriptional expression levels of atrogin-1 and MuRF1 which are muscle-degrading proteins were checked.

Specifically, C2C12 muscle cells were treated with squalene and cultured for 12 hours while inducing differentiation, by performing the same method as in the above <Example 1>. The resulting cells were obtained. Total RNA was isolated from the obtained cells using a TRIzol reagent (Invitrogen, Carlsbad, Calif., USA). The isolated total RNA was quantitated using NanoDrop 1000 (Thermo Fisher Scientific Inc., Waltham, Mass., USA). The quantified 16 μL of RNA was synthesized into cDNA using Reverse Transcriptase Premix (ELPISBIOTECH. INC) and a PCR machine (Gene Amp PCR System 2700; Applied Biosystems, Foster City, Calif., USA) under a condition at 42° C. for 55 minutes and 70° C. for 15 minutes. 3 μL of cDNA out of the synthesized cDNA, specific primers (BIONEER CORPORATION, Daejeon, Korea) as shown in the following [Table 1], and a PCR premix (ELPISBIOTECH. INC) were mixed, and PCR was performed by repeating 30 cycles of 95° C. for 30 seconds, 60° C. for 1 minute, and 72° C. for 1 minute. After PCR amplification, cDNA was separated by electrophoresis on 1.5% agarose gel, and cDNA bands were identified using a G:BOX EF imaging system (Syngene).

TABLE 1

| Primer name | Direction | Sequence | SEQ ID NO |
|---|---|---|---|
| Atrogin-1_F | Forward | 5′-CAGTGATCCATTCTGTTCATCCTTG-3′ | SEQ ID NO: 1 |
| Atrogin-1_R | Reverse | 5′-TTATTTCCAGCCAAATGGAGAGAGA-3′ | SEQ ID NO: 2 |
| MuRF1_F | Forward | 5′-TCTGCACTTAGAACACATAGCAGAG-3′ | SEQ ID NO: 3 |
| MuRF1_R | Reverse | 5′-TCTCCTTCTTCATTGGTGTTCTTCT-3′ | SEQ ID NO: 4 |
| β-Actin_F | Forward | 5′-CAGCTCAGTAACAGTCCGCC-3′ | SEQ ID NO: 5 |
| β-Actin_R | Reverse | 5′-TCACTATTGGCAACGAGCGG-3′ | SEQ ID NO: 6 |

As a result, as illustrated in FIG. 3, it was identified that mRNA expression levels of atrogin-1 and MuRF1, which are muscle-destroying proteins, in C2C12 muscle cells are decreased due to treatment with squalene. This means that the squalene of the present invention has a superior ability to inhibit muscle protein degradation in muscle cells.

### Example 4

Effect of Squalene which Improves Muscular Strength and Regenerates Damaged Muscles

<4-1> Induction of Muscle Atrophy and Muscle Damage Through Immobilization

Seven-week-old male rats (C57BL/6J; DBL Co., Ltd.) were purchased as experimental animals, and experiments were conducted. All animals were kept at the Yonsei Laboratory Animal Research Center (YLARC, Seoul, Korea), and an environment under which the animals were kept was maintained at a temperature of 23±2° C. and a relative humidity of 55±10%. Before starting experiments, a total of 28 rats were randomly assigned to 7 rats per group, and the groups were divided into a normal group, an immobilized group, 100 mg/kg/day of squalene-administered group (squalene 100-administered group), and 200 mg/kg/day of squalene-administered group (squalene 200-administered group). After one week of adaptation, anesthesia was induced by intraperitoneal injection of 325 mg/kg of tribromoethanol (Sigma-Aldrich). After anesthesia, for the rats belonging to the immobilized group, the squalene 100-administered group, and the squalene 200-administered group, the gastrocnemius muscle of the right hindlimb and the right sole were stapled using a skin stapler (UNIDUS Corporation, North Chungcheong Province, Korea) so that the right hindlimb did not move, and this state was maintained for a week. One week later, the staples fixed on the gastrocnemius muscle and the sole were removed and squalene was orally administered at a concentration of 100 mg/kg and 200 mg/kg on a daily basis for one week. The normal group and the immobilized group were orally administered saline instead of squalene.

<4-2> Effect of Squalene which Improves Muscular Strength

After the oral administration period was completed in the above <Example 4-1>, muscular strength of the rats was measured using a muscular strength meter (Columbus Instruments International, Columbus, Ohio, USA). All forelimbs and hindlimbs of the rat were grasped on a grid. Then, the rat was held by the tail and pulled with the same force. Five measurements were consecutively performed, and a maximum value was selected.

As a result, as illustrated in FIG. 4, it was identified that muscular strength is significantly decreased (^{##}P<0.01) in the immobilized group as compared with the normal group, and it was identified that muscular strength is restored in a concentration-dependent manner due to treatment with squalene (**P<0.01). This means that the squalene of the present invention has a superior ability to increase muscular strength.

<4-3> Effect of Squalene which Increases Muscle Weight

After the measurement of muscular strength in the above <Example 4-2> was completed, the experimental animals were anesthetized by intraperitoneal injection of 325 mg/kg of tribromoethanol (Sigma-Aldrich), and then sacrificed by cardiac puncture. After identifying that heartbeat was stopped, a tibialis anterior muscle of the right hindlimb which was not damaged by the stapler but had not been available was extracted and weighed.

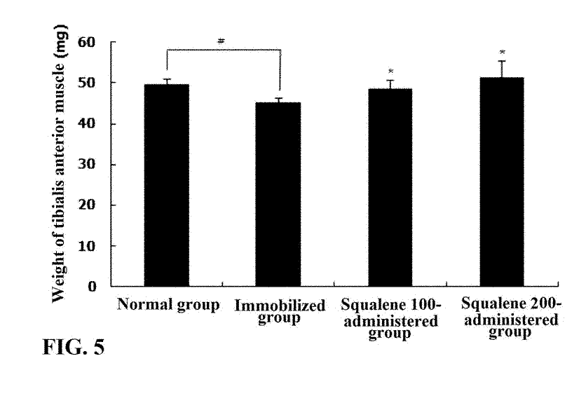

As a result, as illustrated in FIG. 5, it was identified that a weight of the tibialis anterior muscle is significantly decreased (^{#}P<0.05) in the immobilized group as compared with the normal group, and it was identified that a muscle weight is increased in a concentration-dependent manner due to treatment with squalene (*P<0.05). This means that the squalene of the present invention has a superior effect of increasing muscle weight.

<4-4> Effect of Squalene which Increases Myofiber Cross-Sectional Area

A part of the tibialis anterior muscle tissue extracted in the above <Example 4-3> was removed and fixed with 10% formalin to make a paraffin block. After fixation, hematoxylin and eosin staining was performed to measure muscle restoration in terms of histology. The stained tissue was observed with a photochemical microscope (CK40; Olympus Corporation, Tokyo, Japan) equipped with an eXcope T500 camera (DIXI Science, Daejeon, Korea). In addition, myofibers were photographed and a cross-sectional area thereof was measured with an Image J program (National Institutes of Health, Bethesda, ML, USA).

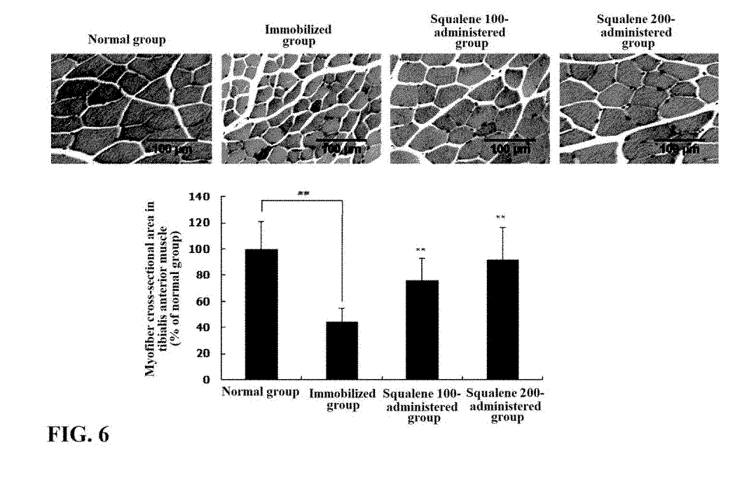

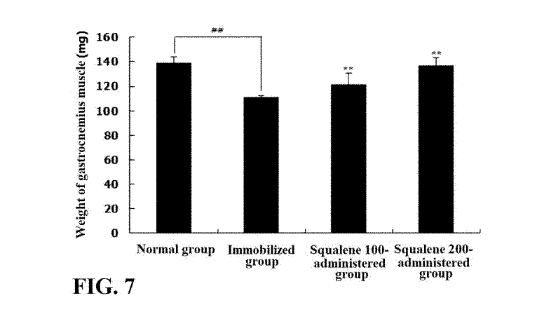

As a result, as illustrated in FIG. 6, it was identified that the myofiber cross-sectional area in the tibialis anterior muscle is significantly decreased (^{##}P<0.01) in the immobilized group as compared with the normal group, and it was identified that the myofiber cross-sectional area is increased in a concentration-dependent manner due to treatment with squalene (**P<0.01). This means that the squalene of the present invention has a superior effect of increasing muscle size.

<4-5> Effect of Squalene which Promotes Regeneration of Damaged Muscles

In the rat from which the tibialis anterior muscle of the right hindlimb was extracted in the above <Example 4-3>, a tibialis anterior muscle of the right hind limb, which was physically directly damaged by the stapler, was extracted and weighed.

As a result, as illustrated in FIG. 7, it was identified that a weight of the tibialis anterior muscle is significantly decreased (^{##}P<0.01) in the immobilized group as compared with the normal group, and it was identified that a muscle weight is increased in a concentration-dependent manner due to treatment with squalene (**P<0.01). This means that the squalene of the present invention exhibits a superior effect of promoting regeneration of damaged muscles.

Hereinafter, preparation examples for medicines, foods, or cosmetics which comprise, as an active ingredient, the squalene according to the present invention will be described. However, such preparation examples are provided merely to specifically describe the present invention and are not intended to limit the present invention. Medicine, food, or cosmetic compositions of Preparation Examples 1 to 3 were prepared according to conventional methods in compliance with the following compositional ingredients and compositional ratios using the squalene having a superior effect of preventing, treating, or ameliorating muscle disease, or regenerating damaged muscles.

### <Preparation Example 1> Preparation of Pharmaceutical Preparations

<1-1> Preparation of Powders

| | |
|---|---|
| Squalene of present invention | 0.1 g |
| Lactose | 1.5 g |
| Talc | 0.5 g |

The above ingredients were mixed, and the mixture was filled in an airtight bag to prepare powders.

<1-2> Preparation of Tablets

| | |
|---|---|
| Squalene of present invention | 0.1 g |
| Lactose | 7.9 g |
| Crystalline cellulose | 1.5 g |
| Magnesium stearate | 0.5 g |

The above ingredients were mixed, and then a direct tableting method was used to prepare tablets.

<1-3> Preparation of Capsules

| | |
|---|---|
| Squalene of present invention | 0.1 g |
| Corn starch | 15 g |
| Carboxycellulose | 4.9 g |

The above ingredients were mixed to prepare powders, and then the powders were filled in hard capsules according to a conventional method for preparing capsules, to prepare capsules.

<1-4> Preparation of Injections

| | |
|---|---|
| Squalene of present invention | 0.1 g |
| Sterilized water for injection | adequate amount |
| pH adjuster | adequate amount |

Injections were prepared to have the above contents of ingredients per ampoule (2 ml) according to a conventional method for preparing injections.

<1-5> Preparation of Liquids

| | |
|---|---|
| Squalene of present invention | 0.1 g |
| Isomerized sugar | 10 g |
| Mannitol | 5 g |
| Purified water | adequate amount |

According to a conventional method for preparing liquids, the respective ingredients were added in purified water and dissolved therein. An adequate amount of a lemon flavor was added therein, and then the above ingredients were mixed. Next, purified water was added to adjust a total amount to 100. Then, a brown bottle was filled with the resultant, and sterilization was performed to prepare liquids.

### <Preparation Example 2> Preparation of Foods

<2-1> Preparation of Flour Foods

0.5 to 5.0 parts by weight of the squalene of the present invention was added to flour and mixed. The mixture was used to prepare bread, cakes, cookies, crackers, and noodles.

<2-2> Preparation of Soups and Gravies

0.1 to 5.0 parts by weight of the squalane of the present invention was added to soups and gravies so as to prepare soups and gravies of meat-processed products and noodles for health promotion.

<2-3> Preparation of Ground Beef

10 parts by weight of the squalene of the present invention was added to ground beef so as to prepare ground beef for health promotion.

<2-4> Preparation of Dairy Products

5 to 10 parts by weight of the squalane of the present invention was added to milk, and the milk was used to prepare various dairy products such as butter and ice cream.

<2-5> Preparation of Health Supplement Foods

| Squalene of present invention | 100 | mg |
|---|---|---|
| Vitamin mixture | adequate amount | |
| Vitamin A acetate | 70 | μg |
| Vitamin E | 1.0 | mg |
| Vitamin B1 | 0.13 | mg |
| Vitamin B2 | 0.15 | mg |
| Vitamin B6 | 0.5 | mg |
| Vitamin B12 | 0.2 | μg |
| Vitamin C | 10 | mg |
| Biotin | 10 | μg |
| Nicotinic acid amide | 1.7 | mg |
| Folic acid | 50 | μg |
| Calcium pantothenate | 0.5 | mg |
| Mineral mixture | adequate amount | |
| Ferrous sulfate | 1.75 | mg |
| Zinc oxide | 0.82 | mg |
| Magnesium carbonate | 25.3 | mg |
| Potassium phosphate monobasic | 15 | mg |
| Calcium phosphate dibasic | 55 | mg |
| Potassium citrate | 90 | mg |
| Calcium carbonate | 100 | mg |
| Magnesium chloride | 24.8 | mg |

For compositional proportions of the above-mentioned vitamin mixture and mineral mixture, ingredients that are relatively suitable for health foods were mixed in a preferred embodiment. However, blending proportions thereof may be changed in a predetermined manner for practicing the present invention. According to a conventional method for producing healthy foods, the above-mentioned ingredients can be mixed, and then granules can be prepared. The granules can be used for preparing health food compositions according to conventional methods.

<2-6> Preparation of Health Beverages

| | |
|---|---|
| Squalene of present invention | 100 mg |
| Citric acid | 100 mg |
| Oligosaccharide | 100 mg |
| Plum concentrate | 2 mg |
| Taurine | 100 mg |
| Purified water | amount to make total of 500 ml |

According to a conventional method for preparing health beverages, the above ingredients were mixed and then the mixture was stirred and heated at 85° C. for about 1 hour. Then, the resulting solution was filtered and brought into a 1-L sterilized container. The container was sealed and sterilized, and refrigerated. The solution was used for preparing a health beverage composition of the present invention.

For the above-mentioned compositional proportions, ingredients that are relatively suitable for favorite beverages were mixed in a preferred embodiment. However, blending proportions thereof may be changed in a predetermined manner for practicing the present invention, depending on regional or national preference such as demanding classes, demanding countries, and intended uses.

### <Preparation Example 3> Preparation of Cosmetic Composition

<3-1> Nourishing Lotion (Milk Lotion)

A nourishing lotion (milk lotion) comprising the squalene of the present invention can be prepared according to a conventional preparation method in the field of cosmetics by performing blending as described in the following [Table 2].

TABLE 2

| | Preparation Example |
|---|---|
| Ingredient for blending | 3-1 (% by weight) |
| Squalene of present invention | 2.0 |
| Squalane | 5.0 |
| Beeswax | 4.0 |
| Polysorbate 60 | 1.5 |
| Sorbitan sesquioleate | 1.5 |
| Liquid paraffin | 0.5 |
| Caprylic or capric triglyceride | 5.0 |
| Glycerine | 3.0 |
| Butylene glycol | 3.0 |
| Propylene glycol | 3.0 |
| Calboxyvinyl polymer | 0.1 |
| Triethanolamine | 0.2 |
| Preservative, pigment, and flavoring agent | adequate amount |
| Purified water | to 100 |

<3-2> Softening Lotion (Skin Lotion)

A softening lotion (skin lotion) comprising the squalene of the present invention can be prepared according to a conventional preparation method in the field of cosmetics by performing blending as described in the following [Table 3].

TABLE 3

| | Preparation Example |
|---|---|
| Ingredient for blending | 3-2 (% by weight) |
| Squalene of present invention | 2.0 |
| Glycerine | 3.0 |
| Butylene glycol | 2.0 |
| Propylene glycol | 2.0 |
| Calboxyvinyl polymer | 0.1 |
| PEG 12 nonylphenyl ether | 0.2 |
| Polysorbate 80 | 0.4 |
| Ethanol | 10.0 |
| Triethanolamine | 0.1 |
| Preservative, pigment, and flavoring agent | adequate amount |
| Purified water | to 100 |

<3-3> Nourishing Cream

A nourishing cream comprising the squalene of the present invention can be prepared according to a conventional preparation method in the field of cosmetics by performing blending as described in the following [Table 4].

TABLE 4

| | Preparation Example |
|---|---|
| Ingredient for blending | 3-3 (% by weight) |
| Squalene of present invention | 2.0 |
| Polysorbate 60 | 1.5 |
| Sorbitan sesquioleate | 0.5 |
| PEG60 hydrogenated castor oil | 2.0 |
| Liquid paraffin | 10 |
| Squalane | 5.0 |
| Caprylic or capric triglyceride | 5.0 |
| Glycerine | 5.0 |
| Butylene glycol | 3.0 |
| Propylene glycol | 3.0 |
| Triethanolamine | 0.2 |
| Preservative | adequate amount |
| Pigment | adequate amount |
| Flavoring agent | adequate amount |
| Purified water | to 100 |

<3-4> Massage Cream

A massage cream comprising the squalene of the present invention can be prepared according to a conventional preparation method in the field of cosmetics by performing blending as described in the following [Table 5].

TABLE 5

| | Preparation Example |
|---|---|
| Ingredient for blending | 3-4 (% by weight) |
| Squalene of present invention | 1.0 |
| Beeswax | 10.0 |
| Polysorbate 60 | 1.5 |
| PEG 60 hydrogenated castor oil | 2.0 |
| Sorbitan sesquioleate | 0.8 |
| Liquid paraffin | 40.0 |
| Squalane | 5.0 |
| Caprylic or capric triglyceride | 4.0 |
| Glycerine | 5.0 |
| Butylene glycol | 3.0 |
| Propylene glycol | 3.0 |
| Triethanolamine | 0.2 |
| Preservative, pigment, and flavoring agent | adequate amount |
| Purified water | to 100 |

<3-5> Pack

A pack comprising the squalene of the present invention can be prepared according to a conventional preparation method in the field of cosmetics by performing blending as described in the following [Table 6].

TABLE 6

| | Preparation Example |
|---|---|
| Ingredient for blending | 3-5 (% by weight) |
| Squalene of present invention | 1.0 |
| Polyvinyl alcohol | 13.0 |
| Sodium carboxymethylcellulose | 0.2 |
| Glycerine | 5.0 |
| Allantoin | 0.1 |
| Ethanol | 6.0 |
| PEG 12 nonylphenyl ether | 0.3 |
| Polysorbate 60 | 0.3 |
| Preservative, pigment, and flavoring agent | adequate amount |
| Purified water | to 100 |

<3-6> Gel

A gel comprising the squalene of the present invention can be prepared according to a conventional preparation method in the field of cosmetics by performing blending as described in the following [Table 7].

TABLE 7

| | Preparation Example |
|---|---|
| Ingredient for blending | 3-6 (% by weight) |
| Squalene of present invention | 0.5 |
| Ethylenediamine sodium acetate | 0.05 |
| Glycerine | 5.0 |
| Calboxyvinyl polymer | 0.3 |
| Ethanol | 5.0 |
| PEG 60 hydrogenated castor oil | 0.5 |
| Triethanolamine | 0.3 |
| Preservative, pigment, and flavoring agent | adequate amount |
| Purified water | to 100 |

The present invention as described above is not limited by the above-described examples and preparation examples. For the present invention, various changes and modifications may be made by those skilled in the art. The present invention may be applied to cosmetics for various uses including other color cosmetics, and may be used for preparing a medicament which can be applied thinly on a human body, that is, an ointment, depending on efficacy thereof. These are included in the spirit and scope of the present invention as defined in the appended claims.

### INDUSTRIAL APPLICABILITY

As described above, the present invention provides a composition for preventing, treating, or ameliorating muscle disease and muscle damage, comprising squalene as an active ingredient. More specifically, the squalene of the present invention can increase expression of proteins which are associated with muscle protein synthesis and muscle mass increase in muscle cells, inhibit, at an mRNA level, expression of enzymes involved in muscle protein degradation, and rapidly restore damaged muscles, and thus exhibits a superior effect on prevention, treatment, or amelioration of muscle disease and muscle damage. Therefore, the squalene of the present invention can be safely used without side effects, and can provide a composition that exhibits a remarkable effect on prevention, treatment, or amelioration of muscle disease and muscle damage, so that the present invention has high industrial applicability.

1. A composition for preventing or treating muscle disease or muscle damage, comprising as an active ingredient:
squalene which is a compound represented by the following [Formula 1]:

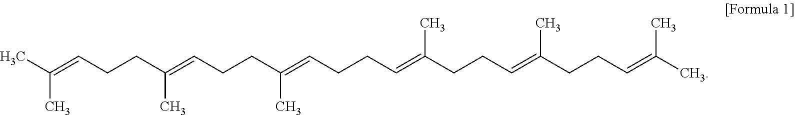 [SMILES: CC(C)=CCC/C(C)=C/CC/C(C)=C/CC/C=C(\C)CC/C=C(\C)CCC=C(C)C]

2. The composition according to claim 1,
wherein the muscle disease is muscle disease caused by decreased muscular function, muscle wasting, or muscle degeneration.

3. The composition according to claim 2,
wherein the muscle disease is any one or more selected from the group consisting of sarcopenia, muscular atrophy, muscular dystrophy, muscle degeneration, and cachexia.

4. The composition according to claim 1,
wherein the muscle damage is exercise-induced muscle damage.

5. The composition according to claim 1,
wherein the muscle damage is selected from the group consisting of muscle strain, muscle rupture, muscle tearing, contusion, distortion, rotator cuff syndrome, and myositis.

6. A food composition for improving muscular function, preventing muscle damage, ameliorating muscle damage, or regenerating muscles, comprising the composition according to claim 1

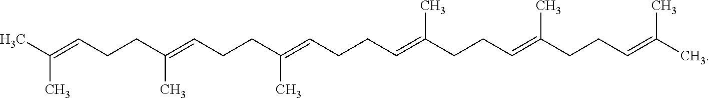 [SMILES: CC(C)=CCC/C(C)=C/CC/C(C)=C/CC/C=C(\C)CC/C=C(\C)CCC=C(C)C]

7. A cosmetic composition for improving muscular function or regenerating muscles, comprising the composition according to claim 1

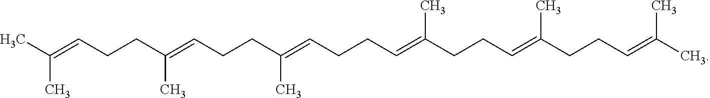 [SMILES: CC(C)=CCC/C(C)=C/CC/C(C)=C/CC/C=C(\C)CC/C=C(\C)CCC=C(C)C]

8. A method for preventing or treating muscle disease or muscle damage, comprising:
a step of administering, to an individual in need thereof, a pharmaceutically effective amount of squalene which is a compound represented by the following [Formula 1]

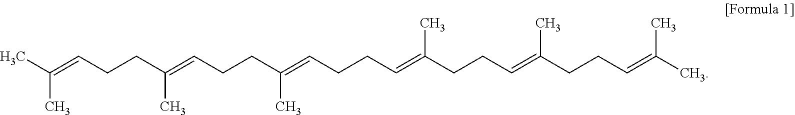 [SMILES: CC(C)=CCC/C(C)=C/CC/C(C)=C/CC/C=C(\C)CC/C=C(\C)CCC=C(C)C]

9. (canceled)

10. A method for improving muscular function or regenerating muscles, comprising:
a step of administering, to an individual in need thereof, a pharmaceutically effective amount of squalene which is a compound represented by the following [Formula 1]:

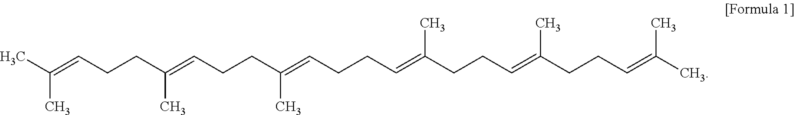 [SMILES: CC(C)=CCC/C(C)=C/CC/C(C)=C/CC/C=C(\C)CC/C=C(\C)CCC=C(C)C]

11. (canceled)

12. (canceled)

13. (canceled)
